SEQUENCE LISTING (24 sequences)

SEQ ID NO: 1 - DNA, 1653 bp, Photinus pyralis
gtcaccgacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 2 - protein, 550 aa, Photinus pyralis
VTDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 3 - DNA, 27 bp, Artificial Sequence
  Primer
attgaattcgtcaccgacgccaaaaac

SEQ ID NO: 4 - DNA, 27 bp, Artificial Sequence
  Primer
attggatccttacacggcgatctttcc

SEQ ID NO: 5 - DNA, 1431 bp, Homo sapiens
atgggcgcgggggtgctcgtcctgggcgcctccgagcccggtaacctgtcgtcggccgcaccgctccccgacggcgcggccaccgcggcgcggctgctggtgcccgcgtcgccgcccgcctcgttgctgcctcccgccagcgaaagccccgagccgctgtctcagcagtggacagcgggcatgggtctgctgatggcgctcatcgtgctgctcatcgtggcgggcaatgtgctggtgatcgtggccatcgccaagacgccgcggctgcagacgctcaccaacctcttcatcatgtccctggccagcgccgacctggtcatggggctgctggtggtgccgttcggggccaccatcgtggtgtggggccgctgggagtacggctccttcttctgcgagctgtggacctcagtggacgtgctgtgcgtgacggccagcatcgagaccctgtgtgtcattgccctggaccgctacctcgccatcacctcgcccttccgctaccagagcctgctgacgcgcgcgcgggcgcggggcctcgtgtgcaccgtgtgggccatctcggccctggtgtccttcctgcccatcctcatgcactggtggcgggcggagagcgacgaggcgcgccgctgctacaacgaccccaagtgctgcgacttcgtcaccaaccgggcctacgccatcgcctcgtccgtagtctccttctacgtgcccctgtgcatcatggccttcgtgtacctgcgggtgttccgcgaggcccagaagcaggtgaagaagatcgacagctgcgagcgccgtttcctcggcggcccagcgcggccgccctcgccctcgccctcgcccgtccccgcgcccgcgccgccgcccggacccccgcgccccgccgccgccgccgccaccgccccgctggccaacgggcgtgcgggtaagcggcggccctcgcgcctcgtggccctgcgcgagcagaaggcgctcaagacgctgggcatcatcatgggcgtcttcacgctctgctggctgcccttcttcctggccaacgtggtgaaggccttccaccgcgagctggtgcccgaccgcctcttcgtcttcttcaactggctgggctacgccaactcggccttcaaccccatcatctactgccgcagccccgacttccgcaaggccttccagggactgctctgctgcgcgcgcagggctgcccgccggcgccacgcgacccacggagaccggccgcgcgcctcgggctgtctggcccggcccggacccccgccatcgcccggggccgcctcggacgacgacgacgacgatgtcgtcggggccacgccgcccgcgcgcctgctggagccctgggccggctgcaacggcggggcggcggcggacagcgactcgagcctggacgagccgtgccgccccggcttcgcctcggaatccaaggtg

SEQ ID NO: 6 - protein, 496 aa, Homo sapiens
MGAGVLVLGASEPGNLSSAAPLPDGAATAARLLVPASPPASLLPPASESPEPLSQQWTAGMGLLMALIVLLIVAGNVLVIVAIAKTPRLQTLTNLFIMSLASADLVMGLLVVPFGATIVVWGRWEYGSFFCELWTSVDVLCVTASIETLCVIALDRYLAITSPFRYQSLLTRARARGLVCTVWAISALVSFLPILMHWWRAESDEARRCYNDPKCCDFVTNRAYAIASSVVSFYVPLCIMAFVYLRVFREAQKQVKKIDSCERRFLGGPARPPSPSPSPVPAPAPPPGPPRPAAAAATAPLANGRAGKRRPSRLVALREQKALKTLGIIMGVFTLCWLPFFLANVVKAFHRELVPDRLFVFFNWLGYANSAFNPIIYCRSPDFRKAFQGLLCCARRAARRRHATHGDRPRASGCLARPGPPPSPGAASDDDDDDVVGATPPARLLEPWAGCNGGAAADSDSSLDEPCRPGFASESKVFASESKVGQHRQGHPENRD

SEQ ID NO: 7 - DNA, 30 bp, Artificial Sequence
  Primer
atagatatcatgggcgcgggggtgctcgtc

SEQ ID NO: 8 - DNA, 30 bp, Artificial Sequence
  Primer
atagaattccaccttggattccgaggcgaa

SEQ ID NO: 9 - DNA, 3084 bp, Homo sapiens
atgggcgcgggggtgctcgtcctgggcgcctccgagcccggtaacctgtcgtcggccgcaccgctccccgacggcgcggccaccgcggcgcggctgctggtgcccgcgtcgccgcccgcctcgttgctgcctcccgccagcgaaagccccgagccgctgtctcagcagtggacagcgggcatgggtctgctgatggcgctcatcgtgctgctcatcgtggcgggcaatgtgctggtgatcgtggccatcgccaagacgccgcggctgcagacgctcaccaacctcttcatcatgtccctggccagcgccgacctggtcatggggctgctggtggtgccgttcggggccaccatcgtggtgtggggccgctgggagtacggctccttcttctgcgagctgtggacctcagtggacgtgctgtgcgtgacggccagcatcgagaccctgtgtgtcattgccctggaccgctacctcgccatcacctcgcccttccgctaccagagcctgctgacgcgcgcgcgggcgcggggcctcgtgtgcaccgtgtgggccatctcggccctggtgtccttcctgcccatcctcatgcactggtggcgggcggagagcgacgaggcgcgccgctgctacaacgaccccaagtgctgcgacttcgtcaccaaccgggcctacgccatcgcctcgtccgtagtctccttctacgtgcccctgtgcatcatggccttcgtgtacctgcgggtgttccgcgaggcccagaagcaggtgaagaagatcgacagctgcgagcgccgtttcctcggcggcccagcgcggccgccctcgccctcgccctcgcccgtccccgcgcccgcgccgccgcccggacccccgcgccccgccgccgccgccgccaccgccccgctggccaacgggcgtgcgggtaagcggcggccctcgcgcctcgtggccctgcgcgagcagaaggcgctcaagacgctgggcatcatcatgggcgtcttcacgctctgctggctgcccttcttcctggccaacgtggtgaaggccttccaccgcgagctggtgcccgaccgcctcttcgtcttcttcaactggctgggctacgccaactcggccttcaaccccatcatctactgccgcagccccgacttccgcaaggccttccagggactgctctgctgcgcgcgcagggctgcccgccggcgccacgcgacccacggagaccggccgcgcgcctcgggctgtctggcccggcccggacccccgccatcgcccggggccgcctcggacgacgacgacgacgatgtcgtcggggccacgccgcccgcgcgcctgctggagccctgggccggctgcaacggcggggcggcggcggacagcgactcgagcctggacgagccgtgccgccccggcttcgcctcggaatccaaggtggtcaccgacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 10 - protein, 1027 aa, Homo sapiens
MGAGVLVLGASEPGNLSSAAPLPDGAATAARLLVPASPPASLLPPASESPEPLSQQWTAGMGLLMALIVLLIVAGNVLVIVAIAKTPRLQTLTNLFIMSLASADLVMGLLVVPFGATIVVWGRWEYGSFFCELWTSVDVLCVTASIETLCVIALDRYLAITSPFRYQSLLTRARARGLVCTVWAISALVSFLPILMHWWRAESDEARRCYNDPKCCDFVTNRAYAIASSVVSFYVPLCIMAFVYLRVFREAQKQVKKIDSCERRFLGGPARPPSPSPSPVPAPAPPPGPPRPAAAAATAPLANGRAGKRRPSRLVALREQKALKTLGIIMGVFTLCWLPFFLANVVKAFHRELVPDRLFVFFNWLGYANSAFNPIIYCRSPDFRKAFQGLLCCARRAARRRHATHGDRPRASGCLARPGPPPSPGAASDDDDDDVVGATPPARLLEPWAGCNGGAAADSDSSLDEPCRPGFASESKVVTDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 11 - DNA, 1239 bp, Homo sapiens
atggggcaacccgggaacggcagcgccttcttgctggcacccaatagaagccatgcgccggaccacgacgtcacgcagcaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttccattgatgtgctgtgcgtcacggccagcattgagaccctgtgcgtgatcgcagtggatcgctactttgccattacttcacctttcaagtaccagagcctgctgaccaagaataaggcccgggtgatcattctgatggtgtggattgtgtcaggccttacctccttcttgcccattcagatgcactggtaccgggccacccaccaggaagccatcaactgctatgccaatgagacctgctgtgacttcttcacgaaccaagcctatgccattgcctcttccatcgtgtccttctacgttcccctggtgatcatggtcttcgtctactccagggtctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagacgttaggcatcatcatgggcactttcaccctctgctggctgcccttcttcatcgttaacattgtgcatgtgatccaggataacctcatccgtaaggaagtttacatcctcctaaattggataggctatgtcaattctggtttcaatccccttatctactgccggagcccagatttcaggattgccttccaggagcttctgtgcctgcgcaggtcttctttgaaggcctatgggaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctg

SEQ ID NO: 12 - protein, 413 aa, Homo sapiens [sequence omitted]

SEQ ID NO: 13 - DNA, 32 bp, Artificial Sequence
  Primer
atagcggccgcatggggcaacccgggaacggc

SEQ ID NO: 14 - DNA, 33 bp, Artificial Sequence
  Primer
atagaattccagcagtgagtcatttgtactaca

SEQ ID NO: 15 - DNA, 2892 bp, Homo sapiens
atggggcaacccgggaacggcagcgccttcttgctggcacccaatagaagccatgcgccggaccacgacgtcacgcagcaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttccattgatgtgctgtgcgtcacggccagcattgagaccctgtgcgtgatcgcagtggatcgctactttgccattacttcacctttcaagtaccagagcctgctgaccaagaataaggcccgggtgatcattctgatggtgtggattgtgtcaggccttacctccttcttgcccattcagatgcactggtaccgggccacccaccaggaagccatcaactgctatgccaatgagacctgctgtgacttcttcacgaaccaagcctatgccattgcctcttccatcgtgtccttctacgttcccctggtgatcatggtcttcgtctactccagggtctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagacgttaggcatcatcatgggcactttcaccctctgctggctgcccttcttcatcgttaacattgtgcatgtgatccaggataacctcatccgtaaggaagtttacatcctcctaaattggataggctatgtcaattctggtttcaatccccttatctactgccggagcccagatttcaggattgccttccaggagcttctgtgcctgcgcaggtcttctttgaaggcctatgggaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctggtcaccgacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 16 - protein, 963 aa, Homo sapiens
MGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSQGRNCSTNDSLLVTDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 17 - DNA, 1398 bp, Homo sapiens
atgaataactcaacaaactcctctaacaatagcctggctcttacaagtccttataagacatttgaagtggtgtttattgtcctggtggctggatccctcagtttggtgaccattatcgggaacatcctagtcatggtttccattaaagtcaaccgccacctccagaccgtcaacaattactttttattcagcttggcctgtgctgaccttatcataggtgttttctccatgaacttgtacaccctctacactgtgattggttactggcctttgggacctgtggtgtgtgacctttggctagccctggactatgtggtcagcaatgcctcagttatgaatctgctcatcatcagctttgacaggtacttctgtgtcacaaaacctctgacctacccagtcaagcggaccacaaaaatggcaggtatgatgattgcagctgcctgggtcctctctttcatcctctgggctccagccattctcttctggcagttcattgtaggggtgagaactgtggaggatggggagtgctacattcagtttttttccaatgctgctgtcacctttggtacggctattgcagccttctatttgccagtgatcatcatgactgtgctatattggcacatatcccgagccagcaagagcaggataaagaaggacaagaaggagcctgttgccaaccaagaccccgtttctccaagtctggtacaaggaaggatagtgaagccaaacaataacaacatgcccagcagtgacgatggcctggagcacaacaaaatccagaatggcaaagcccccagggatcctgtgactgaaaactgtgttcagggagaggagaaggagagctccaatgactccacctcagtcagtgctgttgcctctaatatgagagatgatgaaataacccaggatgaaaacacagtttccacttccctgggccattccaaagatgagaactctaagcaaacatgcatcagaattggcaccaagaccccaaaaagtgactcatgtaccccaactaataccaccgtggaggtagtggggtcttcaggtcagaatggagatgaaaagcagaatattgtagcccgcaagattgtgaagatgactaagcagcctgcaaaaaagaagcctcctccttcccgggaaaagaaagtcaccaggacaatcttggctattctgttggctttcatcatcacttgggccccatacaatgtcatggtgctcattaacaccttttgtgcaccttgcatccccaacactgtgtggacaattggttactggctttgttacatcaacagcactatcaaccctgcctgctatgcactttgcaatgccaccttcaagaagacctttaaacaccttctcatgtgtcattataagaacataggcgctacaagg

SEQ ID NO: 18 - protein, 466 aa, Homo sapiens
MNNSTNSSNNSLALTSPYKTFEVVFIVLVAGSLSLVTIIGNILVMVSIKVNRHLQTVNNYFLFSLACADLIIGVFSMNLYTLYTVIGYWPLGPVVCDLWLALDYVVSNASVMNLLIISFDRYFCVTKPLTYPVKRTTKMAGMMIAAAWVLSFILWAPAILFWQFIVGVRTVEDGECYIQFFSNAAVTFGTAIAAFYLPVIIMTVLYWHISRASKSRIKKDKKEPVANQDPVSPSLVQGRIVKPNNNNMPSSDDGLEHNKIQNGKAPRDPVTENCVQGEEKESSNDSTSVSAVASNMRDDEITQDENTVSTSLGHSKDENSKQTCIRIGTKTPKSDSCTPTNTTVEVVGSSGQNGDEKQNIVARKIVKMTKQPAKKKPPPSREKKVTRTILAILLAFIITWAPYNVMVLINTFCAPCIPNTVWTIGYWLCYINSTINPACYALCNATFKKTFKHLLMCHYKNIGATR

SEQ ID NO: 19 - DNA, 31 bp, Artificial Sequence
  Primer
atagatatcatgaataactcaacaaactcct

SEQ ID NO: 20 - DNA, 30 bp, Artificial Sequence
  Primer
atagaattcccttgtagcgcctatgttctt

SEQ ID NO: 21 - DNA, 3051 bp, Homo sapiens
atgaataactcaacaaactcctctaacaatagcctggctcttacaagtccttataagacatttgaagtggtgtttattgtcctggtggctggatccctcagtttggtgaccattatcgggaacatcctagtcatggtttccattaaagtcaaccgccacctccagaccgtcaacaattactttttattcagcttggcctgtgctgaccttatcataggtgttttctccatgaacttgtacaccctctacactgtgattggttactggcctttgggacctgtggtgtgtgacctttggctagccctggactatgtggtcagcaatgcctcagttatgaatctgctcatcatcagctttgacaggtacttctgtgtcacaaaacctctgacctacccagtcaagcggaccacaaaaatggcaggtatgatgattgcagctgcctgggtcctctctttcatcctctgggctccagccattctcttctggcagttcattgtaggggtgagaactgtggaggatggggagtgctacattcagtttttttccaatgctgctgtcacctttggtacggctattgcagccttctatttgccagtgatcatcatgactgtgctatattggcacatatcccgagccagcaagagcaggataaagaaggacaagaaggagcctgttgccaaccaagaccccgtttctccaagtctggtacaaggaaggatagtgaagccaaacaataacaacatgcccagcagtgacgatggcctggagcacaacaaaatccagaatggcaaagcccccagggatcctgtgactgaaaactgtgttcagggagaggagaaggagagctccaatgactccacctcagtcagtgctgttgcctctaatatgagagatgatgaaataacccaggatgaaaacacagtttccacttccctgggccattccaaagatgagaactctaagcaaacatgcatcagaattggcaccaagaccccaaaaagtgactcatgtaccccaactaataccaccgtggaggtagtggggtcttcaggtcagaatggagatgaaaagcagaatattgtagcccgcaagattgtgaagatgactaagcagcctgcaaaaaagaagcctcctccttcccgggaaaagaaagtcaccaggacaatcttggctattctgttggctttcatcatcacttgggccccatacaatgtcatggtgctcattaacaccttttgtgcaccttgcatccccaacactgtgtggacaattggttactggctttgttacatcaacagcactatcaaccctgcctgctatgcactttgcaatgccaccttcaagaagacctttaaacaccttctcatgtgtcattataagaacataggcgctacaagggtcaccgacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 22 - protein, 1016 aa, Homo sapiens
MNNSTNSSNNSLALTSPYKTFEVVFIVLVAGSLSLVTIIGNILVMVSIKVNRHLQTVNNYFLFSLACADLIIGVFSMNLYTLYTVIGYWPLGPVVCDLWLALDYVVSNASVMNLLIISFDRYFCVTKPLTYPVKRTTKMAGMMIAAAWVLSFILWAPAILFWQFIVGVRTVEDGECYIQFFSNAAVTFGTAIAAFYLPVIIMTVLYWHISRASKSRIKKDKKEPVANQDPVSPSLVQGRIVKPNNNNMPSSDDGLEHNKIQNGKAPRDPVTENCVQGEEKESSNDSTSVSAVASNMRDDEITQDENTVSTSLGHSKDENSKQTCIRIGTKTPKSDSCTPTNTTVEVVGSSGQNGDEKQNIVARKIVKMTKQPAKKKPPPSREKKVTRTILAILLAFIITWAPYNVMVLINTFCAPCIPNTVWTIGYWLCYINSTINPACYALCNATFKKTFKHLLMCHYKNIGATRVTDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 23 - DNA, 30 bp, Artificial Sequence
  Primer
ataggatccatggggcaacccgggaacggc

SEQ ID NO: 24 - DNA, 70 bp, Artificial Sequence
  Primer
ataaagcttaatctcggttctctgggtgaccctgcctatgctgacccagcagtgagtcatttgtactaca

(19) United States
(12) Patent Application Publication
(10) Pub. No.: US 2018/0143213 A1
(43) Pub. Date: May 24, 2018

(54) METHODS FOR THE DETECTION OF ANTIBODIES AGAINST MEMBERS OF THE CARDIAC RECEPTOR FAMILY

(71) Applicant: Lutz Schomburg, Berlin (DE)

(72) Inventors: Lutz Schomburg, Berlin (DE); Waldemar Minich, Berlin (DE); Niels-Peter Becker, Berlin (DE)

(73) Assignee: Lutz Schomburg, Berlin (DE)

(21) Appl. No.: 15/536,610

(22) PCT Filed: Dec. 21, 2015
(86) PCT No.: PCT/EP2015/080732

§ 371 Date: Jun. 16, 2017

(30) Foreign Application Priority Data
Dec. 19, 2014 (DE) 10 2014 226 663.7

(51) Int. Cl. G01N 33/94

(52) U.S. Cl. CPC G01N 33/9433; G01N 2800/32; G01N 33/944

(57) ABSTRACT

The present invention relates to methods for the detection of antibodies against members of the cardiac receptor family; kits for performing the methods of the invention; the use of the methods of the invention for the diagnosis, therapy and/or prophylaxis of one or more diseases, which are related to one or more members of the cardiac receptor family, and the use of the methods of the invention for a) the identification of modulators of the binding properties of antibodies against members of the cardiac receptor family orb) the identification of therapeutic agents for the treatment of one or more of the said diseases.

The present invention relates to methods for the detection of antibodies against members of the cardiac receptor family; kits for performing the methods of the invention; the use of the methods of the invention for the diagnosis, therapy and/or prophylaxis of one or more diseases, which are related to one or more members of the cardiac receptor family, in particular of a disease selected from the group consisting of high blood pressure, dilated cardiomyopathy, glaucoma, chronic fatigue, and dementia; as well as the use of the methods of the invention for a) the identification of modulators of the binding properties of antibodies against members of the cardiac receptor family or b) the identification of therapeutic agents for the treatment of one or more of the above mentioned diseases.

Heart failure (HF) is the pathologic impairment of the heart resulting in an insufficient supply of blood to the body's tissues. Causative factors for HF include a reduced pump function (systolic heart insufficiency) or a defective filling of the heart (diastolic heart insufficiency). HF is the main cause of mortality in the Western civilization and creates enormous financial and social damage. Approximately 2% of the overall adults suffer from HF, with an increasing incidence and prevalence correlating with advancing age (6-10% at the age 65 and older are affected).

Cardiac function is controlled by the autonomic nervous system. Signal transmission is triggered by the cardiac receptors, mainly by adrenergic receptors (ARs) and muscarinic acetylcholine receptors (mAChRs). Adrenergic receptors (ARs) belong to the superfamily of G-protein coupled receptors (GPCRs) targeted by catecholamines, especially adrenaline and noradrenaline. Nine ARs have been characterized in human, divided into three families: al adrenergic receptors (α1ARs), α2 adrenergic receptors (α2ARs) and β adrenergic receptors (βARs). Three subtypes of βARs have been described: β1AR, β2AR and β3AR, all expressed in the human heart. ARs regulate many physiological processes, including pacemaker activity, myocardial contraction and vascular muscle tonus. Of particular clinical relevance are β1AR and β2AR, with β1AR being the predominant subtype in the heart. Hence, β1AR is the main target for the therapeutically active class of compounds called ‘β-blockers’, widely used in the treatment of cardiovascular diseases.

Acetylcholine receptors (AChRs) play also a prominent role beside the ARs. There are two types of AChRs: nicotinic AChRs (nAChRs) and muscarinic AChRs (mAChRs). nAChRs are ionotropic and hence, function as an ion channel themselves. mAChRs are metabotropic receptors (protein-coupled) and belong to the superfamily of GPCRs. mAChRs trigger, e.g., heartbeat and heart pressure. Due to their pharmacological properties, mAChRs are subclassified into five subtypes: M1 to M5. The unevenly numbered mAChRs M1, M3 and M5 are associated with the Gq heterotrimeric G-protein. The evenly numbered mAChRs M2 and M4 are associated with the Gi heterotrimeric G-protein. For example, the activation of M2 leads to a reduced heart rate.

Autoantibodies (“Aab's”) play an important role in the pathogenesis of autoimmune diseases. Recent research results suggest the pathogenic and diagnostic relevance of antibodies (“Ab's”) against cardiac receptors, e.g., a high concentration of Aab's against α1ARs is associated with increased blood pressure. Clinical studies show a significant correlation between β1AR autoantibody (“Aab”) titers of HF patients compared with healthy control. The presence of Aab's against mAChR M2 is associated with an increased risk for cardiomyopathy. Additionally, mAChRs-Aab's were found with a high prevalence in fibrillation patients (Semin. Immunopathol. 2014 May, 36(3), 351-63). Due to the limited availability of high throughput screening methods, the pathogenic relevance of Ab's against members of the cardiac receptor family has not yet been subject to epidemiological investigation and few data are available for a differential diagnosis of heart dysfunction and treatment. Thus, the development of a fast, specific, sensitive, and reliable method for the determination of autoantibodies against particular cardiac (sub-) receptors may play an important role, even more, if such method allows for the determination of cross-reactivity. Such methods would allow early diagnosis or differential diagnosis and may be of particular value in the prevention, treatment, or the monitoring of an autoimmune related disorder related to one or more members of the cardiac receptor family. Furthermore, it is desired to provide a cheap and effective method, which would further allow the identification of modulators (i.e., activators, inhibitors, or molecules that otherwise influence the interaction between an antigen and an antibody) of the interaction, resp., binding of one or more member of the cardiac receptor family with Aab's or a therapeutic agent.

Currently available test methods may not provide the desired specificity, scalability, manageability, sensitivity, and/or may require large amounts of the biological sample. Hence, there is the need to improve the methods and assay technology. The present invention as defined in the claims overcomes the above mentioned problems of the prior art by providing methods exhibiting improved speed, specificity and/or sensitivity, which are further suitable to automatization or high throughput screening. The methods according to instant invention are close to physiological conditions as they allow the antigen to form the correct three-dimensional structure. This is one of the major differences between instant invention and methods using proteins or peptide fragments expressed from bacteria. The methods of the invention are suitable for automatization and the performance in research and/or clinical laboratories. Furthermore, the methods of the invention allow the identification of specific cross-reactivities between pathological or therapeutical antibodies and the members of the cardiac receptor family. Surprisingly, due to the increased sensitivity and/or specificity of the methods of instant invention, the present methods allow the differentiated investigation of interactions, as well as the development of improved approaches and agents in the prophylaxis, diagnosis, and therapy of diseases or disorders, which are related to the members of the cardiac receptor family.

Therefore, it is a first embodiment of instant invention to provide a method of detecting in a sample to be investigated the presence and/or the binding properties of analyte antibodies reactive with one or more antigenic molecules, said method comprising the steps of:
(a) providing one or more first antigenic molecules with which analyte antibodies when present in said sample can interact and which first antigenic molecule is selected from the cardiac receptor family (CRF); and
(b) providing one or more second antigenic molecules with which analyte antibodies when present in said sample can interact and which second antigenic molecule is selected from the CRF; and
(c) contacting said first antigenic molecules as provided by step (a) and said second antigenic molecules as provided by step (b) simultaneously or successively with the sample to be investigated, whereby analyte antibodies when present in said sample can interact with said antigenic molecules so as to form complexes comprising [first antigenic molecule]-[analyte antibody]-[second antigenic molecule]; and
(d1) prior to, or concurrent with, or subsequent to, step (c), providing immobilizing means whereby said first antigenic molecule as present in the said complexes formed in step (c), respectively, as capable to form complexes in step (c) can be immobilized to a solid support prior to, or concurrent with, or subsequent to, step (c); and/or
(d2) prior to, or concurrent with, or subsequent to, step (c), providing second labeling means whereby said first antigenic molecule as present in the said complexes formed in step (c), respectively, as capable to form complexes in step (c) is labeled with said second labeling means prior to, or concurrent with, or subsequent to, step (c); and
(e) prior to, or concurrent with, or subsequent to, step (c), providing first labeling means whereby said second antigenic molecule as present in the said complexes formed in step (c), respectively, as capable to form complexes in step (c) is labeled with said first labeling means prior to, or concurrent with, or subsequent to, step (c); and
(g) detecting the presence of the said complexes formed in or subsequent to step (c) so as to provide indication of analyte antibodies present in said sample, wherein the said first and the said second labeling means are different, and preferably, wherein the use of a radioactive labeling means is excluded.

Another embodiment of the method of the invention comprises the steps of:
(a) providing one or more first antigenic molecules with which analyte antibodies when present in said sample can interact and which first antigenic molecule is selected from the cardiac receptor family (CRF); and
(b) providing one or more second antigenic molecules with which analyte antibodies when present in said sample can interact and which second antigenic molecule is selected from the CRF; and
(c) contacting said first antigenic molecules as provided by step (a) and said second antigenic molecules as provided by step (b) simultaneously or successively with the sample to be investigated, whereby analyte antibodies when present in said sample can interact with said antigenic molecules so as to form complexes comprising [first antigenic molecule]-[analyte antibody]-[second antigenic molecule]; and
(d1) prior to step (c), providing immobilizing means whereby said first antigenic molecule as present in the said complexes formed in step (c), respectively, as capable to form complexes in step (c) can be immobilized to a solid support prior to step (c); and/or
(d2) prior to step (c), providing second labeling means whereby said first antigenic molecule as present in the said complexes formed in step (c), respectively, as capable to form complexes in step (c) is labeled with said second labeling means prior to step (c); and
(e) prior to step (c), providing first labeling means whereby said second antigenic molecule as present in the said complexes formed in step (c), respectively, as capable to form complexes in step (c) is labeled with said first labeling means prior to step (c); and
(g) detecting the presence of the said complexes formed in or subsequent to step (c) so as to provide indication of analyte antibodies present in said sample,
wherein the said first and the said second labeling means are different, and preferably, wherein the use of a radioactive labeling means is excluded.

In another embodiments of the methods of the invention it is further excluded:
a) the use of a radioactive labeling means and any step including precipitation, or
b) the use of a radioactive labeling means and any step including centrifugation.

According to instant invention, the first and second labeling means are selected to be different and provide distinguishable, resp., distinct signals, preferably, distinctively detectable signals by means of a single measurement of the applicable detection method. In alternative embodiments of the invention, the antigenic moieties within the first and second antigenic molecules are identical or different. In yet another embodiment of the invention, the first and/or second antigenic molecules are embedded in a membrane-like or membrane environment. In still another embodiment of the present invention, the analyte Ab's of the sample to be investigated are Aab's of a specific individual or therapeutic Ab's or diagnostic Ab's, the latter being preferably monoclonal. In yet another embodiment of the methods of the invention the one or more components, which are selected from the first labeling means, the second labeling means, the immobilization means and the modulators, are provided prior to contacting the said antigens with said analyte Ab's, i.e., the one or both antigens are labeled, resp., immobilized, to generate the first and second antigenic molecules before the said complexes together with the antibodies are formed.

Another object of the invention is the in vitro use of the methods of the invention for diagnosing in a subject the presence of or disposition to a disease, which is related to the function of a receptor (or part thereof), which is a member of the cardiac receptor family (“CRF”). Yet another object of the invention is the use of the methods and/or kits of the invention for the identification of a pharmaceutically effective compound for the treatment and/or prophylaxis of one or more diseases, which are related to the function of a receptor (or part thereof), which is a member of the CRF, in particular, selected from high blood pressure, dilatative cardiomyopathy, glaucoma, chronic fatigue, and dementia, particularly preferred the use of an automated method, especially a high throughput screening assay (“HTS”).

Preferably, the methods of the present invention are in vitro methods, which may comprise steps in addition to those mentioned above, which may be related to the pretreatment of the sample or the assessment, resp., further processing of the primary or secondary test signals of the method, especially, with respect to the detection of the presence of the said complexes. The methods of the invention may partially or completely performed manually or automated. Optionally, one or more of the steps (a), (b), (c), (c1), (c2), (d), (d1), (d2), (e), (f), and (g), (h), and (i) of the method of the invention may partially or completely be assisted by automation, including suitable robotic and sensory equipment and/or computer implemented processing and/or evaluation of the primary signals. Furthermore, the skilled person is aware, that the methods of the invention preferably require a calibration or standardization of the signal in order to assure the quantification of the detected signals and, resp., the presence of the complexes to be identified, e.g., by means of an internal or external standard, i.e., one or more known quantities of reference compounds (reference Ab's or complexes).

In alternative embodiments of the present invention, the first antigens and the second antigens are identical or not identical (i.e. diverse). In further alternative embodiments of the invention the first and second antigens are diverse and belong to the same or diverse member of the CRF. In another embodiment of the invention, the first and/or the second antigen are embedded in a membrane-like or membrane environment. In a preferred embodiment of the invention the method allows the detection of said Ab's against a member of the CRF in a concentration of about 0.03 to 3 ng/ml, preferably 0.03 to 1 ng/ml, and more preferred 0.03 to 0.1 ng/ml.

In another embodiment of the methods of the invention the one or more first antigenic molecules are provided prior to step (c) in an immobilized form (e.g. coupled to a solid support), preferably prior to the contact with the sample to be analyzed. Optionally, the solid support may be provided in a liquid phase (e.g., as a dispersion, suspension, or colloid), alternatively, it can be provided as, e.g., a microtiter plate or any material suitable for affinity chromatography. Such immobilized one or more first antigenic molecules are subsequently contacted with the sample to be analyzed either simultaneously or consecutively, and with the one or more second antigenic molecules.

The immobilized one or more first antigenic molecules when contacted with the said sample may form intermediate complexes comprising [first antigenic molecule]-[analyte antibody] wherein the one or more first antigenic molecule is immobilized to a solid support and the thus formed immobilized intermediate complex is subsequently contacted with the one or more second antigenic molecules, present in solution, so as to form the hitherto described complexes comprising [first antigenic molecule]-[analyte antibody]-[second antigenic molecule] directly or indirectly immobilized to a solid support via the first antigenic molecule.

In another embodiment of the methods of the invention the one or more first antigenic molecules, which are tagged by a second labeling means, are provided prior to step (c). In another embodiment of the methods of the invention, the one or more first antigenic molecules are immobilized to a solid support and are provided prior to step (c). In another embodiment of the methods of the invention, the one or more second antigenic molecules, which are tagged by a first labeling means, are provided prior to step (c). In another embodiment of the methods of the invention both the one or more first antigenic molecules, which are immobilized to a solid support, and the one or more second antigenic molecules, which are tagged by a first labeling means, are provided prior to step (c). In another embodiment of the methods of the invention both the one or more first antigenic molecules, which are tagged by a second labeling means, and the one or more second antigenic molecules, which are tagged by a first labeling means, are provided prior to step (c). In yet another embodiment of the methods of the invention, the one or more first antigenic molecules are immobilized to a solid support and the one or more second antigenic molecules are tagged by a first labeling means, wherein the said second antigenic molecules are provided in a solution. In yet another embodiment of the methods of the invention, the one or more first antigenic molecules are immobilized to a solid support and the one or more second antigenic molecules are tagged by a first labeling means, wherein both said first and second antigenic molecules are provided in a solution. Another embodiments of the methods of the present invention allow the direct monitoring of the interaction of (i) said analyte antibodies present in the sample and (ii) said one or more first antigenic molecules and (iii) said one or more second antigenic molecules as provided, by employing an assay signal detection technology known in the art (e.g., non-competitive or competitive assays), for example, of the sandwich type or RET (resonance energy transfer) type, whereas the latter assay type does not require immobilization and/or separation of the said first antigenic molecules from the liquid phase. Yet another embodiment of the methods of the present invention allow for the identification of modulators, which are capable to interact with the complexes formed in step (c) and/or which are capable to interfere with the complex formation according to step (c) as defined in the method of detecting analyte antibodies according to the present invention. Accordingly, another embodiment of the methods of the present invention further comprises step (f) prior to, or concurrent with, or subsequent to, step (c), providing one or more compounds to be tested for their capability to interact with the complexes formed in step (c) and/or capable to interfere with the complex formation according to step (c) and contacting said test compounds simultaneously or successively with the said sample, the said one or more first antigenic molecules, and/or the said one or more second antigenic molecules prior to, or concurrent with, or subsequent to step (c), or contacting said test compounds simultaneously or successively with the said complexes formed in or subsequent to step (c). In another embodiment of the methods according to the present invention the one or more modulators (i.e. “compounds to be tested for their capability to interact with said complexes or complex formation”) are provided prior to step (c) of the method of detecting antibodies of instant invention. In still other embodiments of the methods of the present invention one or more of the said means selected from the group consisting of the said immobilizing means, the said second labeling means, and the said first labeling means are provided prior to step (c) of the method of detecting autoantibodies according to the present invention.

In a further embodiment the present invention provides a kit, which is useful for performing any of the methods according to the present invention comprising (a) one or more first antigenic molecules selected from the CRF as defined in one or more of the methods according to the invention; (b) one or more second antigenic molecules selected from the CRF as defined in one or more of the methods according to the invention; (c₁) immobilization means as defined in one or more of the methods according to the invention and/or (c₂) second labeling means as defined in one or more of the methods according to the invention; and (d) first labeling means as defined in one or more of the methods according to the invention, and, optionally, one or more analyte antibodies, which are reactive with the one or more first and second antigenic molecules as defined in one or more of the methods according to the invention. In another embodiment the kit according to the present invention comprises (a) said first antigenic molecules labeled with a second labeling means, or (a) said first antigenic molecules immobilized to a solid support; and (b) said second antigenic molecules labeled with a first labeling means.

In still other embodiments the present invention provides the use of any of the methods or of the kits according to the invention for the diagnosis of the presence, onset, or prevalence of a disease or dysfunction related to one or more members of the cardiac receptor family and/or for the identification of a pharmaceutically effective compound for the treatment and/or prophylaxis of a disease or dysfunction related to one or more members of the cardiac receptor family.

Yet another subject of the invention is the use of one or more members selected from the group of the cardiac receptor family (CRF), which are tagged with two or more different labeling means, which are preferably distinguishably detectable, for the performance of a method for the identification of one or more antibodies against one or more members of the CRF, the manufacturing of kits for performing the said methods, and their diagnostic or therapeutic use and their use for the identification of therapeutically effective agents in the treatment of a disease or dysfunction related to one or more members of the CRF, especially of a disease selected from the group consisting of high blood pressure, dilated cardiomyopathy, glaucoma, chronic fatigue, and dementia.

Generally, all terms and phrases used in this application shall have the meaning of the general knowledge of the person skilled in the art. However, the following preferred definitions of some terms and phrases may further specify the invention:

The terms “polypeptide” and “protein” are used synonymously.

The term “sample” according to the present invention has the meaning of “sample to be analyzed”, which essentially comprises a liquid, suspension or dispersion, preferably of biological and/or chemical-synthetic origin. The sample can be obtained by well known techniques and may consist of isolated body fluids such as blood, plasma, serum, cerebrospinal fluid, saliva, urine, seminal liquid, tear fluid and others. The “sample” may further comprise hair, nail clippings, faeces or other excrement, isolated cells, cell homogenates, tissue homogenates, or organ homogenates obtained from an animal (e.g., mouse, rat, guinea pig, dog, pig, primates) or of human origin. Tissue samples or organ samples may be obtained from any tissue or organ by, e.g., biopsy or smear. Separated cells may be obtained from the body fluids or the tissues or organs by separating techniques such as centrifugation or cell sorting. Preferably, cell samples, tissue samples or organ samples are obtained from those cells, tissues or organs which express, contain, accumulate, concentrate or produce the antigens, analyte antibodies, or modulators referred to herein. The sample may have been subject to treatment and/or modification, known to the person skilled in the art in order to allow storage or further processing in a method of the invention. For example, the person skilled in the art knows that the sample may be diluted in an appropriate buffer, or, if the sample is derived from urine, any biotin contained in the sample need to be removed in order to avoid interference with accurate biotin determination, if biotin is used as a label means in the method of the invention. Preferably, the sample may be selected from an individual suspected or assumed onset or presence of a disease or dysfunctional condition (or where such condition shall be monitored), which is related to the regulation of the cardiac function or to the blood pressure, or related diseases (e.g., glaucoma, dementia, chronic fatigue or cardiac arrhythmia). In one embodiment of the invention, the sample may be a reference and comprises a known kind and amount of analyte antibodies and/or one or more modulators, preferably, a known kind and amount of both analyte antibodies and one or more modulator. This may be of particular relevance for the identification of modulators as herein described.

Generally, the term “antigenic molecule” according to the present invention means a synthetically obtained compound comprising the tagged (immobilized and/or labeled) antigen and with which an analyte antibody can interact with and which is capable of binding an (one or more) analyte antibody to form specific complexes comprising [analyte antibody-antigenic molecule].

The term “antigen” according to the present invention means any molecule from the group of the cardiac receptor family, subunits, peptides or fragments thereof, with which the analyte antibody can interact with and which is capable of binding an (one or more) analyte antibody to form specific complexes comprising [analyte antibody-antigenic molecule]. The antigen may be natural or synthetic and modifications thereto are preferably such as to not detrimentally affect the binding properties in the methods according to the present invention.

The term “analyte antibody” according to the present invention means any antibody capable of binding to an (one or more) antigen which is a member of the CRF, respectively, capable of binding to one or more of the receptors selected from the group consisting of adrenergic receptors (AR) and acetylcholinergic receptors (ACR), whose presence is being quantitatively and/or qualitatively analyzed by the methods of the invention as further outlined herein, such as alpha1AR, alpha2AR, betaAR, nicotinic ACR, muscarinic ACR, and subunits, peptides, fragments and/or variants thereof. Specific examples of members of the CRF are: alpha1A AR, alpha1B AR, alpha1D AR, alpha2A AR, alpha2B AR, alpha2C AR, bets1 AR, beta2 AR, beta3 AR, nicotinic neuronal ACR, nicotinic muscular ACR, muscarinic M1 ACR, muscarinic M2 ACR, muscarinic M3 ACR, muscarinic M4 ACR, muscarinic M5 ACR. According to the invention, orthologous or paralogous sequences can be suitable as well.

The term “analyte antibody” further means a monoclonal antibody, a polyclonal antibody, a single chain antibody, a bispecific antibody or diabody, a bivalent antibody, a multispecific antibody, a synthetic antibody, an aptamer, a spiegelmer, a human or humanized antibody, and fragments or variants thereof as well, such as, e.g., Fab-, Fv- or scFv-fragments, or a chemically modified derivative of any of these, e.g., antibody-drug conjugates, domain antibodies, nanobodies or antibody mimetics (DARPins ‘designed ankyrin repeat proteins’). In specific embodiments of the present invention the term “analyte antibodies” means endogenous autoantibodies, therapeutic antibodies and/or diagnostic antibodies.

The term “member of the cardiac receptor family”, respectively, “cardiac receptor family” (CRF) according to the present invention means any polypeptide selected from the group consisting of adrenergic receptors (AR) and acetylcholinergic receptors (ACR), such as alpha1AR, alpha2AR, betaAR, nicotinic ACR, muscarinic ACR, and subunits, peptides, fragments and/or variants thereof, especially alpha1A AR, alpha1B AR, alpha1D AR, alpha2A AR, alpha2B AR, alpha2C AR, beta1 AR, beta2 AR, beta3 AR, nicotinic neuronal ACR, nicotinic muscular ACR, muscarinic M1 ACR, muscarinic M2 ACR, muscarinic M3 ACR, muscarinic M4 ACR, muscarinic M5 ACR, including subunits, variants, analogues, derivatives, fragments, orthologous and paralogous sequences thereof, which comprise a binding domain for an antibody, more preferred such molecules of natural origin. Preferably, the CRF is of animal (e.g., mouse, rat, guinea pig, dog, pig, primates) or human origin, more preferred human.

In further alternative embodiments of the methods according to the invention, the term “CRF” shall exclusively mean: a) an adrenergic receptor (AR); b) an acetylcholinergic receptor (ACR); c) an adrenergic alpha1 receptor; d) an adrenergic alpha2 receptor; e) an adrenergic beta receptor; f) a nicotinic acetylcholinergic receptor; g) a muscarinic acetylcholinergic receptor; or in each case its subunit, variant, analog, derivative or fragment thereof, which comprise a binding domain for an antibody, more preferred such molecules of natural origin. Preferably, the CRF is of animal (e.g., mouse, rat, guinea pig, dog, pig, primates) or human origin, more preferred human, whereas in yet other embodiments, the afore-mentioned definition refers to only the first antigenic molecule.

Suitable polypeptides and their corresponding genes, which encode members of the CRF, are well known to the skilled person in the art. Members of the CRF are commercially available from recombinant sources (e.g. from R&D Systems, Inc., Minneapolis, Minn. 55413, USA, OriGene Technologies, Inc., Rockville, Md. 20850, USA) and are well known from protein and nucleic acid sequence databases, such as, e.g., EMBL, Genbank and others. Currently available database accession numbers for members of the CRF are given for the human species at the specific receptor proteins, however, the present invention shall not be understood to be limited thereto.

The term “adrenergic receptor” (“AR”) according to the present invention means any isolated polypeptide having a naturally occurring amino acid sequence or any variant thereof. The amino acid sequences and gene sequences encoding AR are well known to the skilled person, e.g., from entries in sequence databases such as UniProtKB, or http://www.rcsb.org. The following sequences are exemplified:
a) α1-adrenergic receptors (α1ARs):
   P35348 (ADA1A_HUMAN); P35368 (ADA1B_HUMAN); P25100 (ADA1D_HUMAN);
b) α2-adrenergic receptors (α2ARs):
   P08913 (ADA2A_HUMAN); P18089 (ADA2B_HUMAN); P18825 (ADA2C_HUMAN);
c) β-adrenergic receptors (βARs):
   P08588 (ADRB1_HUMAN); P07550 (ADRB2_HUMAN); P13945 (ADRB3_HUMAN).

In still another embodiment of the present invention, AR means an AR variant, which exhibits pathogenic or dysfunctional prevalence in animals (e.g., mouse, rat, guinea pig, dog, pig, primate) or human. In yet another embodiment of the present invention the AR is embedded in a membrane environment.

The term “acetylcholinergic receptor” (“ACR”) according to the present invention means any isolated polypeptide having a naturally occurring amino acid sequence or any variant of a nicotinic (nACRs) or muscarinic (mACRs) ACRs. At present, 17 different nAChR subunits have been identified, which are divided into muscle-type and neuronal-type subunits (CHRNA1; CHRNA2; CHRNA3; CHRNA4; CHRNA5; CHRNA6; CHRNA7; CHRNA8; CHRNA9; CHRNA10; CHRNB1; CHRNB2; CHRNB3; CHRNB4; CHRND; CHRNE; and CHRNG). Of these 17 subunits, α2-α7, and β2-β4 have been identified in humans. The nicotinic ACRs are pentamers of the said subunits, thus, there is a wide range of variations within the present biological nAChRs, which can be divided into muscle-type, ganglion-type, and different CNS-type classes.

The amino acid sequences and gene sequences encoding ACRs are well known to the skilled person, e.g., from entries in sequence databases such as UniProtKB, or http://www.rcsb.org. The following sequences are exemplified:

Muscarinic acetylcholine receptors (mAChRs): P11229 (ACM1_HUMAN); P08172 (ACM2_HUMAN); P20309 (ACM3_HUMAN); P08173 (ACM4_HUMAN); P08912 (ACM5_HUMAN), as well as for the nicotinic acetylcholine receptors (nAChRs), as well as there subunits (UE), e.g. from UniProtKB: α1 (P02708); α2 (Q15822); α3 (P32297); α4 (P43681); α5 (P30532); α6 (Q15825); α7 (P36544); β1 (P11230); β2 (P17787); β3 (Q05901); β4 (P30926); γ (P07510); δ (Q07001); ϵ (Q04844).

In still another embodiment of the present invention, ACR means an ACR variant, which exhibits pathogenic or dysfunctional prevalence in animals (e.g., mouse, rat, guinea pig, dog, pig, primate) or human. In yet another embodiment of the present invention the ACR is embedded in a membrane environment.

The term “variant” according to the present invention means any fragment, analog, derivative, fusion protein, subunit or subunit chain of antigen mentioned before. Preferably, the “variant” may have essentially the same biological properties as the respective polypeptides or proteins mentioned before, preferably with respect to their immunologic properties, and even more preferred with respect to the dysfunctional properties. Moreover, it is to be understood that the term “variant” according to the present invention means any amino acid sequence which differs due to at least one amino acid substitution, modification, deletion and/or addition, wherein the amino acid sequence of the variant is still, preferably, at least 50%, at least 60%, at least 70%, at least 80%, at least 85%, at least 90%, at least 92%, at least 95%, at least 97%, at least 98%, or at least 99% identical with the amino sequence of the original sequence of the respective polypeptide or protein mentioned, preferably over the entire length of the specific polypeptide or protein. Variants may be allelic variants or any other species-specific homologs, paralogs or orthologs. Moreover, the variants referred to herein include fragments of the respective polypeptides or proteins mentioned hereinbefore from the CRF, provided these fragments have essentially the same biological or pathophysiological properties. Furthermore, the variants referred to herein include fusion proteins of the respective polypeptides or proteins mentioned hereinbefore from the CRF with polypeptides, which are suitable as immobilization means, labeling means, or label, provided such fusion protein essentially maintains the same biological, preferably immunological properties of the respective original polypeptide or protein mentioned hereinbefore from the CRF. Variants may further include modifications of the said polypeptides or proteins by glycosylation or any other chemical or enzymatic modification, provided these variants have essentially the same biological, preferably immunological properties as referred to above. The variants according to the invention may include so-called ‘silent’ substitutions, additions are deletions which do not or not substantially alter the biological activity. More particularly, the variants according to the present invention may have been modified with respect to one or more of the amino acid residues, which are substituted by a conserved or non-conserved amino acid residue, preferably a conserved amino acid residue, or such ones in which one or more of the amino acid resides may include a substituted radical. Such variants are deemed to be within the scope of the teachings herein. Most typically, ‘silent’ variants are those that vary by conservative amino acid substitutions. Such substitutions are those that substitute a given amino acid in a polypeptide by another amino acid of similar chemical characteristics. In this regard are understood as conservative substitutions the replacements, one for another, among the small aliphatic amino acids A, V, L and I; among the hydroxyl residues S and T; among the acidic residues D and E; among the amide residues N and Q; among the basic residues K and R; and among the aromatic residues F and Y (according to the single letter code of amino acids of the IUPAC nomenclature). In one embodiment of the present invention, a “variant” shares essentially the same biological properties. In another embodiment of the present invention a “variant” of a member of the CRF is used, which exhibits pathogenic or dysfunctional prevalence, i.e., which is associated with a disease related to the cardiac receptor family in an animal (e.g., mouse, rat, guinea pig, dog, pig, primates) or human subject.

The term “biological properties” according to the present invention means in particular the binding properties of the respective molecule mentioned. In case of a specific member of the CRF it means its ability to form a complex with its biological, resp., (patho-)physiological ligand under suitable conditions, especially one or more molecules selected from adrenaline, noradrenaline, acetylcholine, or other known active transmitters. Optionally, the term “biological properties” may further include certain specific immunological properties of polypeptides, e.g., if they are specifically detectable by the same ELISA method. Particularly, a member of the CRF exhibits essentially the same “biological properties” like a variant thereof, if both (a) can interact with the analyte antibodies, (b) are detectable by the same ELISA method, and/or (c) are detectable by the detection method according to the present invention.

In another preferred embodiment of the invention the one or more first antigenic molecules and the one or more second antigenic molecules are one or more polypeptides selected from the group consisting of α1A-AR, α1B-AR, α1D-AR, α2A-AR, α2B-AR, α2C-AR, β1-AR, β2-AR, β3-AR, nAChR, and mAChR or variants, subunits and fragments thereof.

The term “providing prior to step (c) of the method of detecting autoantibodies” according to the invention means the provision prior to contacting the said antigenic molecules and the said analyte antibodies.

The term “detecting the presence and/or the binding properties” according to the invention means a qualitative and/or quantitative determination of the relative or absolute amount or concentration of the analyte, preferably a quantitative determination. The signal can be obtained directly or indirectly. Direct measuring relates to measuring the amount or concentration of one or more of the reaction educts and/or reaction products based on a signal which is obtained from the one or more reaction educts and/or the reaction products itself/themselves and the intensity of which directly correlates with the number of molecules of the one or more reaction educts and/or the reaction products in the reaction volume. Such a signal may be obtained, e.g., by measuring the intensity or value of a specific physical or chemical property of the one or more reaction educts and/or reaction products. Indirect measuring includes measuring of a signal obtained from a secondary component (i.e. a component not being the reaction educt or reaction product itself) or a biological read out or amplification system, referred to as “label means” in this specification, e.g., of measurable cellular or transmembrane responses, ligands, or enzymatic reaction products, e.g. by means of fluorophors, chromophors, ion concentrations, which can be performed by means of optical, electrical and/or electronical equipment. For measurement of enzymatic reaction products, preferably the amount of substrate is saturating. Optionally, the substrate may also be labeled with a detectable label prior to the reaction. Preferably, the reaction partners are contacted with the substrate for an adequate period of time, which corresponds to the time necessary for a detectable amount of the one or more reaction products to be produced such as a measurable signal. Instead of measuring the amount or concentration of the one or more reaction products, the time necessary for appearance of a given (e.g. detectable) amount or concentration of the one or more reaction products can be measured.

According to the invention, the term “detecting the presence and/or the binding properties” encompasses all means for determining the amount of a reaction educt and/or reaction product known to the skilled person. Said means comprise methods and devices for the performance of immunoassays which may utilize labeled or immobilized molecules in various (e.g., sandwich, competition, or other) assay formats. Said assays are suitable to generate a signal which is indicative for the presence or absence of the reaction educts and/or the reaction products. Moreover, the signal strength can, preferably, be correlated directly or indirectly (e.g., proportional or reverse-proportional) to the amount of reaction educts and/or the reaction products present in the reaction volume. Said methods comprise, preferably, biosensors, optical devices coupled to immunoassays, biochips, and analytical devices such as spectrometers or chromatography devices. Further methods include ELISA-based methods using, optionally pre-treated or pre-coated, micro-plates, micro-arrays, tube-arrays or chips, fully-automated or robotic immunoassays (available, e.g., by Roche-Elecsys™, Abbott-AxSYM™ or Brahms Kryptor™ analyzer systems). Preferably, “detecting the presence and/or the binding properties” comprises the steps which will allow bringing the reaction partners together for an adequate period of time to obtain a detectable signal.

The term “binding” according to the present invention includes both covalent and non-covalent binding. The term “specific binding” means a binding affinity of at least 3 times higher, preferably of at least 10 times higher and more preferably of at least 50 times higher than the binding affinity to other molecules. In another embodiment of the present invention the term “binding” shall mean the binding of binding partners in an in vitro binding assay under suitable conditions, preferably under conditions according to the assay manufacturer's instructions or according to the methods essentially as defined hereinafter in the Example section. Generally, “binding” means a binding affinity (K_{D}—which means the quotient of dissociation constant to association constant) of about 10⁻¹⁴ M to 10⁻⁷ M, preferably of about 10⁻¹³ M to 10⁻⁹ M.

The term “modulator” according to the invention means any biological or chemical compound, a macromolecule (e.g. larger than about 5 kDa), a small molecule (e.g., smaller than about 5 kDa or even smaller than about 800 Da), an isolated or purified compound or a mixture thereof, a crude extract or homogenates of selected cells, tissue or organ origin, a natural compound or a compound of synthetic origin comprising one or more peptides, polypeptides (e.g., polyclonal or monoclonal antibodies, including single chain antibodies, diabodies, multispecific antibodies, humanized antibodies, hybrid antibodies, or fragments thereof, such as Fv, Fab and F(ab)₂ fragments), aptamers, spiegelmers, nucleic acids, and/or small molecules.

The term “antigenic molecule embedded in a membrane environment” according to the invention means that the said antigenic molecule—which may be derived from a transmembrane protein—is provided in a model membrane or another amphiphilic entity, which includes any suitable synthetic, natural or artificial environment of a, e.g., cellular, membrane, vesicular, micellar or liposomal structure, whereby the said antigenic molecule maintains its functional and/or structural integrity and is able to interact with the aforementioned analyte antibodies and thus allows the said complex formation.

Assay methods for proteins embedded in a membrane environment are well known to the skilled person, e.g., from technologies and methods which use artificial or biomimetical membranes or lipid bilayers (herein “model membranes”). Model membranes are widely used for investigating the properties of membrane proteins and many of them are suitable for the performance of the methods of the present invention, particularly, if denaturation of the said antigenic molecule shall be avoided.

The term “model membrane” according to the invention means any liposome or vesicle, e.g., artificially generated vesicles comprising one or more lipid layers in spherical geometry. Such liposomes and vesicles are also used as vehicles for the transport of lipids, proteins and small molecules and may be used in the administration of pharmaceuticals. Vesicles or liposomes can be prepared by disrupting biological membranes from selected cells cultures, tissues, organs, or subcellular structures (e.g. nucleus, Golgi, endoplasmatic reticulum, mitochondria), e.g., by sonication and/or extrusion and subsequent self-reassembling of the lipid structures, whereby labeling is possible, e.g., with dyes, polypeptides or other labeling means. Further “model membranes” may comprise lipid bilayers which have been synthetically assembled in vitro. They can be made from one or more synthetic and/or natural lipids. The term “model membranes” may further include, e.g., black lipid membranes (BLM), vesicles, lipid bilayers, liposomes, micelles, bicelles, hybrid bilayers (comprising a hydrophobic monolayer and a lipid monolayer), and nanodiscs, which may be anchored, supported or tethered to a solid phase or solid substrate, and which may, optionally, be provided with a spacer or cushion (e.g., polyethylene glycols, oligonucleotides, peptides, polypeptides (e.g., streptavidin) or hydrogels) which may allow to maintain a distance of the membrane and/or the aforementioned antigenic molecule to the solid substrate. Preferably, the spacer or cushion is a hydrophilic molecule. In contrast to a vesicle or a cell membrane, the aforementioned supported bilayer may have a planar structure sitting on a solid support. Therefore, only the upper face of the bilayer is exposed to the solution. For example, the preparation of proteoliposomes is known from patent application EP 1992688 A1, disclosing liposome preparation in examples 1 to 20.

The term “micelles” according to the invention means another type of model membranes without a lipid bilayer. In aqueous solutions, micelles are assemblies of amphipathic molecules (e.g., detergents) with their hydrophilic parts exposed to the polar solvent and their hydrophobic parts in the center. Micelles are able to solubilize membrane proteins by partially embedding them and shielding their hydrophobic surfaces from a polar solvent. The term “bicelles” means still another type of model membranes which are typically made of two lipids, one of which forms a lipid bilayer while the other is an amphipathic, micelle-like assembly shielding the bilayer center from surround solvent molecules. The term “nanodisc” means a segment of a lipid bilayer embedded in an amphipathic protein, a lipid or detergent layer coat. Membrane proteins can also be integrated and solubilized by nanodiscs.

The term “disease related to the cardiac receptor family” (“DRCRF”) according to the invention means any disorder or dysfunction which is related to one or more members of the CRF, preferably a polypeptide which is an AR or ACR as defined above, more preferably referring to one, two, or three of the said members of the CRF. The term “DRCRF” according to the invention preferably means a disorder of permanent or intermediary nature, which is selected from the group consisting of hypertensive dysfunction, high blood pressure, dilated cardiomyopathy, glaucoma, chronic fatigue, dementia, and autoimmune disorders, especially a said disorder of permanent nature, and most preferred one of the said disorders of autoimmune origin.

The term “immobilizing means” according to the invention means any reagent and/or method, which is suitable to immobilize the said first antigenic molecules to a solid support according to the knowledge of the skilled person, preferably maintaining its structural and/or functional integrity. With respect to the kind of the solid support and conditions employed in accordance with the present invention, they do not differ from conventionally used materials, methods, and conditions employed in known immunoassay techniques. A solid support for use according to the present invention can comprise an ELISA plate as currently employed in known ELISA techniques, or may employ any other suitable support for use in the present invention, such as micro-titer plates (having 96, 384, 1536, or 3456 wells or more) or parts thereof, tubes, particles, magnetic beads, nitrocellulose, chip technology or the like. Materials suitable as solid support which can be used in accordance with the teachings of the present invention are well known in the art and include, e.g., commercially available column materials, polystyrene beads and other carriers, latex beads, magnetic beads, colloidal metal, glass surfaces, silanylated surfaces, and silicon surfaces and chips for use in protein microchip technologies, nitrocellulose carriers, cellulose carriers, membranes, model membranes, stabilized liposomes or cells (e.g., duracytes™), wells, resp., surfaces of reaction trays, vessels or microtiter plates, plastic tubes etc.

The said first antigenic molecules may be immobilized to any carrier which is known to the skilled person. Examples of such carriers are inert materials such as glass, polystyrene, polyvinyl chloride, polypropylene, polyethylene, polycarbonate, dextran, nylon, amylose, natural and modified cellulose, polyacrylamide, agarose, magnetite, and gold. The carrier can be either soluble or insoluble, in case of an insoluble carrier; the carrier is a solid or colloid and may, optionally, be provided as suspension. Suitable methods for immobilizing the said antigenic molecules are known and include, but are not limited to ionic, hydrophobic or covalent interactions. It may also be suitable to use said immobilization means in suspension, e.g. hollow microbeads or microspheres, optionally of different kind, optionally labeled, optionally both carrying each different antigenic molecules. Methods for the production of suspensions, e.g., based on solid-phase chemistry and photo-labile protective groups, are known from patent U.S. Pat. No. 5,744,305.

The term “labeling” according to the present invention means direct or indirect labeling. Direct labeling involves coupling of the label (“tag”) directly (covalently or non-covalently) to the molecule to be labeled. Indirect labeling involves binding (covalently or non-covalently) of a second ligand to the molecule to be labeled. Such second ligand specifically binds to the molecule to be labeled (e.g., with an at least 3-fold higher, preferably at least 10-fold, and more preferred at least 50-fold higher affinity) under assay conditions. Said second ligand may also be coupled with a suitable label means and/or may bind a third ligand binding to the second ligand. The use of a second or higher order ligand may be used to increase or amplify the signal. Suitable second and higher order ligands may include antibodies, secondary antibodies, and the well-known streptavidin-biotin system (Vector Laboratories, Inc.). Furthermore, the molecule to be labeled or the substrate may also be “tagged” with one or more tags/labels known in the art. Such tags may then be targets for higher order ligands. Suitable tags include biotin, digoxygenin, His-Tag, Glutathion-S-Transferase, FLAG-tag (N-DYKDDDDK-C), green fluorescence protein (GFP), myc-tag, influenza A virus haemagglutinin (HA), maltose binding protein, and others. In the case of a peptide or polypeptide, the tag is generally located at or close to the N-terminus and/or C-terminus.

Furthermore, the molecule to be labeled or the substrate may also be provided with a suitable “spacer” molecule known in the art in order to avoid in case of bulky molecules any steric limitations with respect to the binding properties due to spatial constrictions.

The term “first labeling means” according to the invention means a direct or indirect detectable labeling means, preferably selected from the group of enzymes, radioactive or isotopes, dyes or their precursors for chemoluminescence, bioluminescence, fluorescence, and magnetic tags (e.g. “magnetic beads”, including paramagnetic and superparamagnetic labels). The “first labeling means” is detectable by an appropriate detection method known in the art. Suitable labels may further include gold particles, latex beads, acridinium ester, luminol, and ruthenium. Enzymatically active labels include, e.g., horseradish peroxidase, alkaline phosphatase, beta-galactosidase, luciferase, and derivatives thereof. Suitable substrates for direct or indirect detection include di-amino-benzidine (DAB), 3, 3′-5, 5′-tetramethylbenzidine, NBT-BCIP (4-nitro blue tetrazolium chloride and 5-bromo-4-chloro-3-indolyl-phosphate, CDP-Star™ (Amersham Biosciences), ECL™ (Amersham Biosciences) and others known in the art. A suitable enzyme-substrate combination may result in the increase or decrease of a colored reaction product or educt (chromophor, fluorescence, chemo- or bioluminescence), which can be detected by known methods (e.g., using a photometer, a photo-multiplier, and a light-sensitive film or camera system). The same principles apply for the quantification when measuring the endpoint, performance or velocity of an enzymatic reaction.

Known fluorescence labels include fluorescent dyes and proteins (such as GFP and its derivatives), Cy3, Cy5, Texas Red, fluorescein, and the Alexa dye series (e.g. Alexa 568), and quantum dots. Many suitable fluorescence labels are commercially available. Examples of fluorescent proteins include, but are not limited to, green, yellow, cyan, blue, and red fluorescent proteins.

Suitable chemoluminescence or bioluminescence labels may include prokaryotic (e.g., bacterial lux-encoded) or eukaryotic (e.g., firefly luc-encoded) luciferases, as well as variants possessing varied or altered optical properties, such as luciferases that emit different colors of light, e.g., derived from Photinus pyralis, from the sponge Suberities domuncula, and the Mycena fungi. Furthermore, photoproteins, e.g., calcium-activated photoproteins and their specifically designed variants may be suitable, which emit light typically in the range of 200 to 1100 nm, or in the visible spectrum (i.e., 350 to 800 nm), e.g., obelin from the marine polyp Obelia longissima, or Aequorin, e.g., from the luminescent jellyfish Aequorea victoria or from other organisms may be suitable, optionally in a membrane.

Suitable radioactive labels include ³⁵S, ¹²⁵I, ³²P, ³³P and other nuclids. A radioactive label can be detected by any method known and appropriate, e.g. a light-sensitive film or a phosphor imager.

Suitable detection methods for performing the invention also include precipitation (particularly immunoprecipitation), electrochemiluminescence (electrically generated chemiluminescence), biolominescence, RIA (radioimmunoassay), ELISA (enzyme-linked immunosorbent assay), sandwich enzyme immune tests, sandwich immunoassays (ECLIA), dissociation-enhanced lanthanide fluorescent Immunoassay (DELFIA™, PerkinElmer Inc., USA), scintillation proximity assay (SPA), turbidimetry, nephelometry, latex-enhanced turbidimetry or nephelometry, latex agglutination assay, or solid phase immune assays, preferably precipitation is excluded.

Further methods known in the art (such as gel electrophoresis, 2D gel electrophoresis, SDS polyacrylamid gel electrophoresis (SDS-PAGE), Western Blotting, and mass spectrometry) can optionally be used in combination with the labeling or other detection methods as described above.

The term “second labeling means” according to the invention means any directly or indirectly detectable labeling means selected from the group of enzymes, radioactive or isotopes, dyes or their precursors for chemoluminescence, bioluminescence, fluorescence, and magnetic tags (e.g. “magnetic beads”, including paramagnetic and superparamagnetic labels). Preferably, the “first labeling means” is a non-radioactive label, more preferred, the “first labeling means” and the “second labeling means” are non-radioactive. The use of the term “second labeling means” means that the “second labeling means” is not identical with the “first labeling means”. The skilled person knows how to select first and second labeling means to ensure that signals from the first and second labeling means are distinguishably detectable in the detection method according to the invention. Suitable second labeling means are detectable by an appropriate detection method known in the art, e.g., based on the resonance energy transfer (RET) principle. Preferably, “second labeling means” include labeling means suitable for fluorescence resonance energy transfer (FRET), bioluminescence resonance energy transfer (BRET), or chemoluminescence resonance energy transfer (CRET) as known to the skilled person. When selecting a suitable second labeling means the skilled person considers the kind of the first labeling means in order to ensure that the RET signal is detectable and distinguishable. For example, RET signal generation and selection of suitable labeling means may provide additional information about the kind and kinetics of complex formation and the structural features of the complexes formed.

### Sequences:

Seq. ID No. 1 LUC DNA sequence

Seq. ID No. 2 LUC Protein sequence

Seq. ID No. 3 Primer P1 Luc Fw

Seq. ID No. 4 Primer P2 Luc Rv

Seq. ID No. 5 cDNA B1

Seq. ID No. 6 AA B1

Seq. ID No. 7 Primer P3 B1 Fw

Seq. ID No. 8 Primer P4 B1 Rv

Seq. ID No. 9 cDNA B1-Luc

Seq. ID No. 10 AA B1-Luc

Seq. ID No. 11 cDNA B2

Seq. ID No. 12 AA B2

Seq. ID No. 13 Primer P5 B2 Fw

Seq. ID No. 14 Primer P6 B2 Rv

Seq. ID No. 15 cDNA B2 Luc

Seq. ID No. 16 AA B2 Luc

Seq. ID No. 17 cDNA M2

Seq. ID No. 18 AA M2

Seq. ID No. 19 Primer P7 M2 Fw

Seq. ID No. 20 Primer P8 M2 Rv

Seq. ID No. 21 cDNA M2 Luc

Seq. ID No. 22 AA M2 Luc

Seq. ID No. 23 Primer P9 B2 2b5 Bac Fw

Seq. ID No. 24 Primer P10 B2 2b5 Bac Rv

The present invention shall be illustrated by the following examples and comparison examples, which do not to limit the scope of the invention.

### Experimental Procedures:

### Materials:

DNA primers were obtained from Life Technologies (Carlsbad, Calif., USA); pSP-luc+NF vector was obtained from Promega GmbH (Mannheim, Germany); pIRESneo vector was obtained from Clontech (Palo Alto, Calif., USA); vector pFastBac1 and High Five™ insect cells were obtained from Invitrogen (Carlsbad, Calif. USA); Polystyrene tubes coated with PGA14 antibody (Selenotest LIA) were obtained from ICI immunochemical intelligence GmbH (Berlin, Germany). If not otherwise stated, other reagents and chemicals were obtained from Sigma-Aldrich Chemie GmbH (Munich, Germany) or Merck KGaA (Darmstadt, Germany); enzymes were obtained from Promega (Madison Wis., USA) or New England Biolabs (Ipswich, Mass., USA).

### EXAMPLE 1

### Construction of Fusion Proteins

### EXAMPLE 1A

### Construction of Beta1-Adrenergic Receptor-Luciferase-Fusion Protein

The DNA (Seq. ID No. 1) encoding amino acids 2-551 of the firefly luciferase (Seq. ID No. 2 on plasmid pSP-luc+NF) was amplified by PCR (polymerase chain reaction') using primers P1 (Seq. ID No. 3) and P2 (Seq. ID No. 4) containing EcoRI and BamHI restriction sites, respectively. Plasmid pIRESneo was digested with EcoRI and BamHI restriction endonucleases; the obtained fragment was replaced by the DNA encoding firefly luciferase obtained from the aforementioned PCR, thus resulting in plasmid pIRESneo-Luc. The cDNA (Seq. ID No. 5) encoding amino acids 1-469 of the human beta 1 adrenergic receptor (Seq. ID No. 6) was amplified by PCR using primers P3 (Seq. ID No. 7) and P4 (Seq. ID No. 8) containing EcoRV and EcoRI restriction sites, respectively. pIRESneo-Luc was digested with EcoRV and EcoRI restriction endonucleases and the obtained fragment was replaced by the DNA sequence encoding human beta 1 adrenergic receptor obtained from the previous PCR resulting in vector pIRESneo-B1-Luc containing Seq. ID No. 9 encoding the labelled fusion protein Seq. ID No. 10.

### EXAMPLE 1B

### Construction of Beta2-Adrenergic Receptor-Luciferase Fusion Protein

The cDNA (Seq. ID No. 11) encoding amino acids 1-413 of the human beta 2 adrenergic receptors (Seq. ID No. 12) was amplified by PCR using primers P5 (Seq. ID No. 13) and P6 (Seq. ID No. 14) containing Not1 and EcoR1 restriction sites, resp.. pIRESneo-Luc was digested with NotI and EcoRI restriction endonucleases and the obtained fragment was replaced with the DNA sequence encoding beta 2 adrenergic receptor obtained from the previous PCR resulting in vector pIRESneo-B2-Luc containing Seq. ID No. 15 encoding the labelled fusion protein Seq. ID No. 16.

### EXAMPLE 1C

### Construction of M2 Muscarinic Receptor-Luciferase Fusion Protein

The cDNA (Seq. ID No. 17) encoding amino acids 1-466 of the M2 muscarinic receptor (Seq. ID No. 18) was amplified by PCR using primers P7 (Seq. ID No. 19) and P8 (Seq. ID No. 20) containing EcoRV and EcoR1 restriction sites, respectively. Plasmid pIRESneo-Luc was digested with EcoRV and EcoR1 restriction endonucleases and the obtained fragment was replaced with the DNA sequence encoding the M2 muscarinic receptor obtained from the previous PCR resulting in vector pIRESneo-M2-Luc containing Seq. ID No. 21 encoding the labelled fusion protein Seq. ID No. 22.

### EXAMPLE 1D

### Construction of a Fusion Protein of the Beta2-Adrenergic Receptor and the Epitope Recognized by PGA14 Antibodies

The cDNA (Seq. ID No. 11) encoding amino acids 1-413 of the beta2-adrenergic receptor (Seq. ID No. 12) was amplified by PCR using primers P9 (Seq. ID No. 23) and P10 (Seq. ID No. 24) containing BamHI and HindIII restriction sites, respectively (P10 containing the coding sequence for the 16 amino acid epitope recognized by PGA14 antibodies). pFastBac1 vector was digested with BamHI and HindIII restriction endonucleases and the obtained fragment was replaced by the DNA sequence encoding the fusion protein of beta2 adrenergic receptor and the PGA14 epitope obtained from the aforementioned PCR resulting in vector pFastBac1-B2-PGA14tag.

### EXAMPLE 2

### Manufacturing of B1-Luc, B2-Luc and M2-Luc Producing Cells

### EXAMPLE 2A

### Manufacturing of B1-Luc, B2-Luc and M2-Luc Producing HEK 293

HEK 293 cells were grown in DMEM-F12 supplemented with 10% fetal bovine serum at 5% CO₂ and 37° C. HEK 293 cells were transfected with one of the plasmids pIRESneo-B1-Luc, pIRESneo-B2-Luc or pIRESneo-M2-Luc using FuGENE6 transfection reagent (obtained from Roche Deutschland Holding GmbH, Grenzach-Wyhlen, Germany) according to the manufacturer's instruction. 48 hours after transfection, the selection was started with 0.8 mg/ml G418 (Gibco™ BRL, Invitrogen). Stable clones expressing high levels of fusion protein were selected.

### EXAMPLE 2B

### Manufacturing of B1-Luc, B2-Luc and M2-Luc Cell Extracts

Confluent HEK293 cells (producing either B1-LUC, B2-LUC or M2-LUC) grown in a 75 cm² plate were harvested and resuspended in PBS. Cells were washed by centrifugation with PBS. The obtained cells were lysed in lysis buffer (50 mM Tris-HCl pH 7.5; 100 mM NaCl; 10% glycerol; 1% triton X-100). The suspension was centrifuged, the supernatant was removed from debris and stored at −80° C.

### EXAMPLE 2C

### Manufacturing of Recombinant Baculovirus Expressing the Fusion Protein B2-PGA14Tag

The B2-PGA14tag sequence obtained from example 1D was transferred to bacmid DNA by site-specific recombination in bacteria. The bacmid was then used to generate a fully recombinant baculovirus in SD insect cells according to the protocols of the manufacturer (Bac-to-Bac expression system manual, Invitrogen).

### EXAMPLE 2D

### Manufacturing of B2-PGA14Tag Fusion Protein

Suspensions of High Five™ insect cells were grown in Express Five™ serum-free medium to density of 2×10⁶ cells/ml. Cells were then infected (transduced) with recombinant B2-PGA14tag-baculovirus at a multiplicity of infection (MOI) of 1. 72 hours post-infection the cells were harvested and the extract was collected and stored at −80C.° as described in Example 2B.

### COMPARISON EXAMPLE 3

### Immunoprecipitation Assay for B2 Autoantibodies (B2-aAb's)

The B2-Luc cell extract obtained from example 2B was diluted 20-fold with buffer containing 50 mM Tris-HCl pH 7.5, 100 mM NaCl, 1% Triton X-100, 10% glycerol, 5 mg/ml BSA. 100 μl of the diluted extract (about 10⁷ RLU) was mixed with 10 μl of a sample (serum probe) and incubated overnight at 4° C. Immune complexes were subsequently precipitated by addition of 10 μl of 10% protein A-sepharose (POROS™, Life Technologies) suspension in the same buffer for 1 h at room temperature with shaking. The Protein A-sepharose was precipitated and washed 3 times with 1 ml washing buffer (50 mM Tris-HCl, pH 7.5, 100 mM NaCl, 0.1% Triton X-100). Finally, luciferase activity of the precipitated immune complexes was measured in a Berthold luminometer (AutoLumat Plus LB 953) for 10 sec. Results were expressed as RLU bound. Table 1 shows the activities of B2-aAb positive sera and B2-aAb negative sera.

### EXAMPLE 4

### Bridge Assays

### EXAMPLE 4A

### Bridge Assay for the Detection of B2-aAb

Polystyrene tubes coated with PGA14 antibody (ICI immunochemical) were incubated overnight at 4° C. with 200 μl of SF6 insect cell medium containing B2-PGA14tag. After B2-PGA14tag immobilization the tubes were washed twice with 1 ml of buffer 20 mM Tris-HCl pH 7.5, 50 mM NaCl, 10% glycerol. Then each tube was incubated overnight at 4C.° with a mixture of 100 μl of the same buffer containing 10 mg/ml BSA and 100 μl of a sample (serum probe). Tubes were washed twice with 1 ml of the same buffer and incubated overnight at 4C.° with 200 μl of B2-Luc obtained as described above in example 2B, diluted in the same buffer with BSA (about 40×10⁶ RLU of luciferase activity). After incubation tubes were washed four times and luciferase activity was measured in a Berthold luminometer (AutoLumat Plus LB 953) for 10 sec. The results shown in Table 1 are expressed as RLU (relative light units) bound and are compared with the results obtained from the conventional assay of Example 3.

TABLE 1
Signals obtained from positive and negative sera in a conventional
immunoprecipitation (Example 3) and the assay of the invention
(Example 4).

| | | Bridge- | | |
|---|---|---|---|---|
| | Precipitate | Assay | Precipitate | Bridge-Assay |
| Table 1 | Example 3 | Example 5 | Example 3 | Example 5 |
| [RLU] | Positive Sera | Positive Sera | Negative Sera | Negative Sera |
| 1 | 82348 | 33734 | 3358 | 553 |
| 2 | 60722 | 45749 | 4835 | 340 |
| 3 | 87350 | 49528 | 3289 | 464 |
| 4 | 69774 | 38763 | 5210 | 533 |
| 5 | 124829 | 96762 | 4427 | 748 |
| 6 | 174171 | 69349 | 4008 | 643 |
| 7 | 105881 | 58823 | 4159 | 462 |
| 8 | 133034 | 95768 | 2776 | 668 |
| 9 | 172382 | 73908 | 4858 | 587 |
| 10 | 375973 | 187864 | 4324 | 523 |
| 11 | | | 3787 | 573 |
| 12 | | | 3389 | 640 |
| 13 | | | 3104 | 707 |
| 14 | | | 4349 | 747 |
| 15 | | | 2898 | 621 |
| 16 | | | 4341 | 602 |
| 17 | | | 2790 | 883 |
| 18 | | | 3464 | 751 |
| 19 | | | 2916 | 511 |
| 20 | | | 2720 | 704 |
| 21 | | | 2200 | 843 |
| Mean | 138646 | 75025 | 3676 | 624 |
| Average | | | | |
| (arithm.) | | | | |
| Control | | | 128 | 624 |

### EXAMPLE 4B

### Detection Limit of the Assay for B2-aAb from Human Serum

Each 3 B2-aAb positive and negative sera were diluted in 20 mM Tris-HCl, pH7.5, 50 mM NaCl, 10% glycerol, 10 mg/ml BSA. The assay was performed as described in Example 4A. Background signal was obtained by buffer only.

TABLE 2
Dilution of B2-aAb positive and negative sera

| Table 2 | | | | |
|---|---|---|---|---|
| [RLU] | undiluted | 1:3 | 1:10 | 1:40 |
| Positive Sera | 86342 | 31807 | 7324 | 1751 |
| | 71164 | 21127 | 8632 | 1097 |
| | 34843 | 15402 | 2584 | 956 |
| Negative Sera | 937 | 678 | 478 | 328 |
| | 848 | 652 | 392 | 442 |
| | 1021 | 748 | 507 | 382 |
| Background | 368 | | | |

### EXAMPLE 4C

### Retrieval Rate of B2-aAb in Human Sera

Three human B2-aAb positive sera (P1-P3) were mixed 1:1 (K1-K3) with negative sera and analyzed as described in Example 4A (Table 3).

TABLE 3
Retrieval rate of B2-aAb

| Table 3 | [RLU] |
|---|---|
| P1 | 86342 |
| K1 | 937 |
| P1 + K1 | 51486 |
| P2 | 71164 |
| K2 | 848 |
| P2 + K2 | 32659 |
| P3 | 34843 |
| K3 | 1021 |
| P3 + K3 | 13286 |

### EXAMPLE 4D

### Direct Coating of the Antigen to Plastic Surface

Three human B2-aAb positive sera and three B2-aAb negative sera were analyzed as described in Example 4A, though with the difference, that the B2-PGA14tag fusion complex from Example 2D was incubated prior to detection with direct uncoated polystyrene tubes (ICI immunochemical intelligence GmbH, Berlin). Controls were analyzed exactly as described in 4A (Table 4).

TABLE 4
Effect of tube coating

| Table 4 | [RLU] |
|---|---|
| P1 | 427 |
| P2 | 365 |
| P3 | 429 |
| K1 | 508 |
| K2 | 337 |
| K3 | 488 |
| Control P1 | 69754 |
| Control K1 | 953 |
| Control without serum | 392 |

### EXAMPLE 4E

### Specific Detection

Three human B2-aAb positive sera and three B2-aAb negative sera were analyzed as described in Example 4A, with the difference, that the assay was performed with 1:500 diluted HRP-coupled rabbit anti-human IgG (DIANOVA, Hamburg Germany) instead of the B2-Luc extract from Example 2B (Table 5).

TABLE 5
Effect of the method of detection

| | RLU |
|---|---|
| P1 | 2632746 |
| P2 | 2357224 |
| P3 | 2468791 |
| K1 | 2586937 |
| K2 | 2703475 |
| K3 | 2638210 |
| Control P1 | 69754 |
| Control K1 | 953 |
| Control without serum | 392 |

### EXAMPLE 4F

### Detection Limit of anti-B2 Antibodies

Known amounts of Antibodies against B2 (sc-569 Santa Cruz, Calif., USA) were diluted in buffer (20 mM Tris-HCl, pH 7.5, 50 mM NaCl, 10% glycerin, 10 mg/ml BSA), as shown in Table and analyzed as described in Example 4A.

TABLE 6
Detection Limit of Anti-B2 Antibodies

| Table 6 | |
|---|---|
| Antikörper [ng/ml] | RLU |
| 0 | 24973 |
| 1 | 24396 |
| 2 | 25762 |
| 5 | 28067 |
| 10 | 33372 |
| 20 | 45107 |
| 50 | 76020 |
| 100 | 93872 |
| 200 | 186542 |
| 500 | 391738 |
| 1000 | 557850 |
| 2000 | 948346 |
| 5000 | 1707022 |
| 10000 | 2389831 |
| 20000 | 2867797 |

### EXAMPLE 4G

### Hetero-bridge

Three B2-aAb positive sera were analyzed as described in Example 4A. Detection of the B2-aAb-crossreactivity was performed using incubation with either 200 μL of the diluted B2-Luc extract, the diluted B1-Luc extract or the diluted M2-Luc extract from Example 2B (40×10⁶ RLU luciferase activity of B1 and B2, 0,5×10⁶ RLU luciferase activity of M2) in buffer with BSA at 4° C. over night (Table 7).

TABLE 7
Crossreactivity of B2-aAb and B1 or M2 receptors

| | B2-receptor- | B2-receptor- | B2-receptor- |
|---|---|---|---|
| | 14PGA + | 14PGA + | 14PGA + |
| | B2-receptor Luc | B1-receptor Luc | M2-receptor Luc |
| Positive Sera | 79856 | 12043 | 401 |
| | 83198 | 3756 | 998 |
| | 61461 | 8237 | 247 |
| Negative Sera | 537 | 923 | 269 |
| | 601 | 754 | 393 |
| | 723 | 968 | 344 |

### EXAMPLE 5

### Clinical Relevance of B2-aAb Level in Humans

Table 8 shows the mean average values (and standard deviation) of the weight (adiposity and diabetes-risk) and age of patient sera exhibiting the presence of B2-aAb in humans.

TABLE 8

| | B2-aAb positive sera | B2-aAb negative |
|---|---|---|
| | (n = 13) | sera (n = 80) |
| Weight [kg] | | |
| Mean average | 77.2 +/− 7.4 | 70.7 +/− 11.4 |
| (arithm.) | | |
| Age (years) | | |
| Mean average | 77.3 +/− 5.5 | 85.0 +/− 5.5 |
| (arithm.) | | |

1. A method of detecting in a sample to be investigated the presence and/or the binding properties of analyte antibodies reactive with one or more antigenic molecules, said method comprising the steps of:
(a) providing one or more first antigenic molecules selected from the cardiac receptor family (CRF); and
(b) providing one or more second antigenic molecules selected from the CRF; and
(c) contacting said first antigenic molecules as provided by step (a) and said second antigenic molecules as provided by step (b) simultaneously or successively with the sample to be investigated, wherein analyte antibodies when present in said sample can interact with said antigenic molecules so as to form complexes comprising:
[first antigenic molecule]-[analyte antibody]-[second antigenic molecule]; and
(d1) prior to, or concurrent with, or subsequent to, step (c), immobilizing the one or more first antigenic molecules using a first immobilizing means to a solid support such that the complexes as formed in step (c) are immobilized; and/or
(d2) prior to, or concurrent with, or subsequent to, step (c), labeling said one or more first antigenic molecules with a second labeling means such that the complexes as formed in step (c) are labeled with the second labeling means; and
(e) prior to, or concurrent with, or subsequent to, step (c), labeling said one or more second antigenic molecules with a first labeling means such that the complexes as formed in step (c) are labeled with the first labeling means; and
(g) detecting or quantifying the presence of complexes [first antigenic molecule]-[analyte antibody]-[second antigenic molecule], formed in or subsequent to step (c), so as to provide indication of analyte antibodies present in said sample.

2. The method of claim 1, further comprising:
(f) prior to, or concurrent with, or subsequent to, step (c), providing a reference sample comprising at least one compound;
(h) contacting said reference sample with said sample, said one or more first antigenic molecules, said one or more second antigenic molecules or said complexes; and
(I1) determining that at least one of the at least one compound is a modulator that is configured to decrease or increase the affinity of said one or more first antigenic molecules or said one or more second antigenic molecules with said analyte antibodies, or
(I2) determining that at least one of the at least one compound is not a modulator that is configured to decrease or increase the affinity of said one or more first antigenic molecules or said one or more second antigenic molecules with said analyte antibodies.

3. The method of claim 1, wherein said first antigenic molecules and said second antigenic molecules are identical.

4. The method according to claim 1, wherein said first antigenic molecules and/or said second antigenic molecules are embedded in a membrane environment.

5. The method according to claim 1, wherein the said analyte antibody to be detected in said sample is an endogenous autoantibody or a monoclonal antibody.

6. The method according to claim 1, wherein one or more of the said means selected from the group consisting of said first labeling means, said second labeling means, said immobilization means, and said at least one compound are provided prior to the contacting said antigenic molecules and said analyte antibodies.

7. A kit useful for the performance of the method according to claim 1 comprising:
(a) one or more first antigenic molecules selected from the cardiac receptor family as defined in claim 1;
(b) one or more second antigenic molecules selected from the cardiac receptor family as defined in claim 1;
(c1) immobilization means as defined in claim 1 and/or
(c2) second labeling means as defined in claim 1; and
(d) first labeling means as defined in claim 1.

8. The kit of claim 7, wherein
(a) the one or more said first antigenic molecules are labeled with a the second labeling means; and
(b) the said one or more second antigenic molecules are labeled with a the first labeling means.

9. The kit of claim 7, wherein
(a) the said one or more first antigenic molecules are immobilized to a solid support; and
(b) the one or more said second antigenic molecules are labeled with a the first labeling means.

10. The method according to claim 1, further comprising:
diagnosing a presence or onset of a disease related to the cardiac receptor family based upon the detection of step (g).

11. The use of the method according to claim 1, further comprising:
identifying a pharmaceutically effective compound for treatment and/or prophylaxis of a disease related to the cardiac receptor family based upon the detection of step (g).

12. A method of using the kit according to claim 7, comprising:
diagnosing a presence or onset of a disease related to the cardiac receptor family.

13. A method of using the kit according to claim 7, comprising:
identifying a pharmaceutically effective compound for the treatment and/or prophylaxis of a disease related to the cardiac receptor family.
